(19) United States
(12) Patent Application Publication
(10) Pub. No.: US 2003/0128849 A1
(43) Pub. Date: Jul. 10, 2003

(54) Acoustic anti-transient-masking transform system for compensating effects of undesired vibrations and a method for developing thereof

(76) Inventor: Ronald L. Meyer, Parker, TX (US)

Correspondence Address: HITT GAINES & BOISBRUN P.C., P.O. BOX 832570, RICHARDSON, TX, 75083, US

(21) Appl. No.: 10/337,696

(22) Filed: Jan. 7, 2003

Related U.S. Application Data
(60) Provisional application No. 60/346,590, filed on Jan. 7, 2002.

(51) Int. Cl. A61F 11/06; G10K 11/16; H03B 29/00; H04R 3/02

(52) U.S. Cl. 381/73.1; 381/71.2

(57) ABSTRACT

The present invention provides a method of developing an acoustic anti-transient-masking transform and an acoustic anti-transient-masking transform system for compensating effects of undesired vibrations impinging an audio component. The present invention also provides a method of compensating an audio signal for effects of undesired vibrations. In one embodiment, the method includes providing an impulse type signal to an undesired vibration compensated version of the audio component and an uncompensated version of the audio component. The method further includes computing a difference impulse response between a first sampled output from the compensated version and a second sampled output from the uncompensated version, and converting the difference impulse response to a signal representing the acoustic anti-transient-masking transform.

### CROSS-REFERENCE TO PROVISIONAL APPLICATION

The This Application claims priority from provisional application 60/346,590 entitled “Mechanical Vibration And Group Delay Effects on Recorded/Reproduced Audio Frequency Program Material,” to Ronald L. Meyer, filed on Jan. 7, 2002, which is commonly assigned with the present invention and incorporated herein by reference as if reproduced herein in its entirety.

### CROSS-REFERENCE TO RELATED APPLICATIONS

This application is related to U.S. patent application Ser. No. ______, entitled “MICROPHONE SUPPORT SYSTEM” to Ronald L. Meyer, filed on Jan. 2, 2003, which is commonly assigned and co-pending with the present invention and is incorporated herein by reference as if reproduced herein in their entirety.

### TECHNICAL FIELD OF THE INVENTION

The present invention is directed, in general, to an audio component and, more specifically, to a method of developing an acoustic anti-transient-masking transform and an acoustic anti-transient-masking transform system for compensating effects of undesired vibrations impinging an audio component.

### BACKGROUND OF THE INVENTION

In modern music performance/recording, mechanical vibration effects on recorded/reproduced audio frequency program material are responsible for perceived (and measured) degradation of the natural transient response of all audio signals captured, stored, replayed, or reproduced by equipment of the prior art. It is a problem that exists at the system level, in all components of the system in one form or another.

The audio industry, since the inception of digital audio in the early 1980s, has faced criticism that digital recordings did not sound as good as their analog counterparts. Indeed, some fine quality recordings were produced by the technology of the late 1950's with analog recording and playback means. This was partially due to the prevalent design techniques used for microphones and microphone stands, along with the materials used in the wiring, and the design of enclosures and chassis. It was also partially due to a more direct signal recording and playback equipment path. That is, there were fewer pieces of equipment to contribute bad effects to the program material, and extra “processing” was not thought of as necessary. Additionally, since the effects of vibration, in some respects, are more detrimental to digital recording and reproduction than to analog processing, the analog recording/playback systems sounded better. In fact, they did indeed capture a better transient response in program material than did the newer digital recordings for reasons disclosed herein.

Microphones are the most susceptible link in the reproduction chain due to their proximity to the original sound source and their natural susceptibility to vibrations. They are self-evidently and inherently, the most sensitive component due to their function, which is to convert airborne vibrations sensed by the element(s) into low level electrical signals for further amplification, storage, analysis, or later reproduction. However, microphone designers have not successfully understood the issue of microphone enclosure vibrations that are also received from the environment, and how they translate into extra modulations which add to the sound already received and are converted by the main microphone sensing element(s). These enclosure-borne vibrations seriously degrade the signal received by the microphone sensing element(s). More specifically, it has been determined that the resonances of various materials comprising the microphone mounting mechanism(s) and stand assembly can cause smeared signal transients.

Common sources of vibration (unwanted inputs to the system) include the program material of interest, “monitoring” equipment used to listen to the desired program material during the recording/reproduction process, internal vibrations generated by power transformers or the mechanisms used to manipulate media (CD or tape transports) used to record or process the desired program material. Even air pressure changes caused by low frequency air handler equipment for HVAC systems (Heating, Ventilation, and Air-Conditioning) can cause vibrations to be introduced into the recorded/amplified program.

The degradation comes in multiple forms, depending on: (a) the type of equipment (analog or digital based signal processing), (b) location in the recording/reproduction chain (microphone or front end processing versus compact disc player playback and power amplifier combination back end processing), and (c) the relative magnitude of the vibration in relation to the signal processing being performed at that stage in the chain. Common effects of the various vibration sources include, but are not necessarily limited to: (a) data clock perturbations in digital systems as a byproduct of the reference crystal vibration (jitter, drift, modulation based on program material), (b) microphonic transfer of vibration to power supply lines which then subsequently modulate the desired program material as a product of amplification, and (c) microphonic transfer of vibration to the microphone electronics through the microphone stand/holder assembly and microphone wiring which then subsequently modulates the desired program material as a by-product of sensing and amplification.

This degradation also affects a human's perception of the transient responses. More specifically, the various vibrations can cause undesired amplitude and phase modulations of an audio signal, causing a perception that the audio signal has slow transient response or lacks clarity. Under the presence of mechanical vibration, or slight variations of component location in relation to one another due to changes in air pressure and/or the surrounding electrical or magnetic fields, amplitude and phase modulation effects are induced onto the signal being processed during: (1) the capture (microphone or sensor pickup), (2) transmission (wiring or cabling through conduction of the induced vibration to the microphone electronics), (3) amplification (either low signal level or high level amplification), (4) encoding/decoding (analog-to-digital or digital-to-analog conversion), and (5) conversion back from electrical signals to sound (speaker, speaker crossovers, external and internal speaker wiring). These vibrations cause a “masking of the transient information” through their modulation of the desired signal. The leading edge of the signal(s) becomes spread out, or compressed, in time as vibrations cause movement of the sensor mechanism, or influence the signal after it has been converted to an electrical signal (microphonics).

When tube circuits were in wide use, the audio industry knew that microphonic effects in tube circuits had an undesirable audible effect on the signal amplified or processed. The audio industry used tube dampers (usually composed of rubber in a circular form) fitted on the tubes themselves in an attempt to limit the effects of vibration on the circuit. After the development of transistors and integrated circuitry, the audio industry thought that undesired vibrations had no effect on the electronic components. However, this same microphonic phenomenon has been found to cause similar effects in transistor circuits that covert or process audio signals, but are manifested in a different manner than tube circuits. For example, the manifestation caused clock circuits to drift or jitter due to vibration effects on a reference crystal. In microphones, the degradation caused by microphonic effects altered the rise time of the sensed signal(s) such that the captured signal no longer mimicked the original signal and, as such, no longer possessed the original response characteristics. For audio program material, the microphonic phenomenon often caused the transient information to have been smeared in time (transient masking). This means that various frequency and phase components of the signal are no longer in their original relation to one another. This also caused a perception that transient responses were blurred to an extent that the recorded or captured sound lost its natural quality and clarity. For additional background information concerning the importance of transient responses and frequency balancing see “Stereophonic and Multi-Channel Amplitude-Panned Localization,” by Ville Pulkki and Matti Karjalainen, Journal of the Audio Engineering Society, Volume 49, No. 9 Pages 739-752 (September 2001), and “Representing Musical Instrument Sounds for Their Automatic Classification,” by Bozena Kostek and Andrzej Czyzewski, Journal of the Audio Engineering Society, Volume 49, No. 9, Pages 768-785 (September 2001), both of which are incorporated by reference in their entirety.

Referring initially to FIG. 1, illustrated is a block diagram of a typical recording/reproduction system that is susceptible to the effects of undesired vibrations. The recording/reproduction system receives desired sounds 102 to record or reproduce through a conventional microphone 110 connected to a microphone cable 114 in a conventional microphone stand 112. The desired sounds 102 may also cause undesired vibrations 104 that impinge upon the microphone 110, the microphone stand 112 and the microphone cable 114. For example, vibrations from a drum may vibrate the microphone 110, the microphone stand 112 and the microphone cable to cause the microphone to covert these vibrations. The undesired vibrations 104 may also be caused by floor borne vibrations, vibrations caused by HVAC systems, vibrations caused by a speaker, and other airborne vibrations.

The microphone 110 is typically the most susceptible audio component in the recording/reproduction chain due to its proximity to the original desired sounds 102 and the susceptibility to vibrations. The microphone 110 by its very nature is the most sensitive audio component due to its function, which is to convert airborne vibrations sensed by the element(s) into low level electrical signals for further amplification, recording, analysis or reproduction. However, microphone designers have not successfully understood the issue of microphone enclosure vibrations that are also received from the environment, and how they translate into extra modulations which add to the sound already received and converted by the main microphone sensing element(s). These enclosure borne vibrations seriously degrade the signal received by the main (desired) sensing element(s), causing smeared signal transients resulting from the resonances of various materials comprising the microphone mounting mechanism(s) and stand assembly 112.

The undesired vibrations 104 can also effect a microphone power supply and/or pre-amplifier 120, an analog-to-digital conversion/storage component 140, a retrieval component 150 and interconnect wiring 130. The undesired vibrations 104 can effect the microphone power supply and the pre-amplifier 120 by vibrating the chassis, the power supply itself and wires entering the component. The analog-to-digital conversion/storage component 140 may be affected by chassis vibrations, wiring connections, and clock jitter and drift. Also, the analog-to-digital conversion/storage component 140 may be susceptible to undesired internal vibrations 106 that occur from components such as motors, fans, crystals or transformers. The retrieval component 150 may be a tape player, CD or DVD player, or hard disk that retrieves a stored audio signal. The retrieval component 150 may also be susceptible to the undesired vibrations 104 which affect the chassis or internal motors. Also, the retrieval component 150 may be susceptible to undesired internal vibrations 106. In addition, any type of audio component may also receive undesired vibrations through an internal power or amplifying transformer. These transformer-borne vibrations can also effect -internal clock circuits employing reference crystals.

The recording/reproduction system may also include a switching and/or amplitude adjustment component 160 that receives audio signals from either the microphone power and pre-amplifier 120 or the retrieval component 150. The recording/reproduction system may also include a signal processing component 170 and an amplification component 180 coupled in series. The switching component 160, the signal processing component 170 and the amplification component 180 are also susceptible to the same types of undesired vibrations 104 and undesired internal vibrations 106 effecting the incoming and outgoing signals, chassis, internal components and interconnect wiring 130 as described above.

The amplification component 180 may also include a digital-to-analog converter that may be affected by clock drift and jitter due to the undesired vibrations 104. The amplification component 180 produces an output signal and sends the output signal via speaker wire 182 to a speaker 190 in a speaker support 192. The speaker 190 then produces the desired sounds in form of airborne vibrations 194. These airborne vibrations 194 may also affect the speaker 190 by producing undesirable vibrations on the speaker support 192. The vibration of the speaker support 192 may then affect the performance of the speaker 190. Thus, every component of the recording/reproduction system is susceptible to the effects of undesired vibrations.

Accordingly, what is needed in the art is way to correct for the effects of undesired vibrations in electronic audio components in their processing and operation.

### SUMMARY OF THE INVENTION

To address the above-discussed deficiencies of the prior art, the present invention provides a method of developing an acoustic anti-transient-masking transform for compensating effects of undesired vibrations impinging an audio component. In one embodiment, the method includes providing an impulse type signal to an undesired vibration compensated version of the audio component and an uncompensated version of the audio component. The method further includes computing a difference impulse response between a first sampled output from the compensated version and a second sampled output from the uncompensated version. Furthermore, the method includes converting the difference impulse response to a signal representing the acoustic anti-transient-masking transform. For purposes of the present invention, the phrase “undesired vibrations” includes undesired vibrations that may occur externally or internally to an audio component.

In another embodiment, the present invention provides an acoustic anti-transient-masking transform system for compensating effects of undesired vibrations impinging an audio component. The system includes a sampling subsystem configured to digitally sample a first output from an undesired vibration compensated version of the audio component in response to an impulse type signal and generate a first sampled output therefrom. The sampling subsystem is further configured to digitally sample a second output from an uncompensated version of the audio component in response to the impulse type signal and generate a second sampled output therefrom. The system further includes a conversion subsystem configured to compute a difference impulse response between the first sampled output and second sampled output, and covert the difference impulse response to a signal representing an acoustic anti-transient-masking transform. For purposes of the present invention, the phrase “configured to” means that the device, the system or the subsystem includes the necessary software, hardware, firmware or a combination thereof to accomplish the stated task.

The present invention, in another embodiment, further provides a method of compensating an audio signal for effects of undesired vibrations. The method includes: (1) determining a type of undesired vibration compensation to apply to the audio signal, (2) retrieving an acoustic anti-transient-masking transform associated with the type of undesired vibration compensation, and (3) multiplying the audio signal by the acoustic anti-transient-masking transform to generate an output signal compensated for effects of the undesired vibrations.

The foregoing has outlined preferred and alternative features of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention. Those skilled in the art should also realize that such equivalent constructions do not depart from the spirit and scope of the invention.

### BRIEF DESCRIPTION OF THE DRAWINGS

For a more complete understanding of the present invention, reference is now made to the following descriptions taken in conjunction with the accompanying drawings, in which:

FIG. 1 illustrates a block diagram of a typical recording/reproduction system that is susceptible the effects of undesired vibrations;

FIG. 2 illustrates a flow diagram of an embodiment of a method of developing an acoustic anti-transient-masking transform for compensating effects of undesired vibrations impinging on an audio component conducted in accordance with the principles of the present invention;

FIG. 3 illustrates an conventional microphone stand uncompensated for undesired vibrations;

FIG. 4 illustrates one embodiment of a microphone support system compensated for undesired vibrations constructed according to the principles of the present invention;

FIG. 5A illustrates one embodiment of a microphone holder compensated for undesired vibrations constructed according to the principles of the present invention;

FIG. 5B illustrates an alternative embodiment of a microphone holder compensated for undesired vibrations constructed according to the principles of the present invention;

FIG. 6A illustrates an uncompensated audio component;

FIGS. 6B and 6C illustrate an audio component compensated for undesired vibrations constructed according to the principles of the present invention;

FIG. 7A illustrates an exemplary time domain graph of the sampled output of the impulse type signal from the compensated microphone support system of FIG. 4;

FIG. 7B illustrates an exemplary time domain graph of the sampled output of the same impulse type signal from an uncompensated version of a microphone stand similar to the one illustrated in FIG. 3;

FIG. 8 illustrates an exemplary time domain graph of the difference impulse response signal between the sampled output from the compensated version and the sampled output from the uncompensated version;

FIG. 9A illustrates an exemplary magnitude graph of an acoustic anti-transient-masking transform derived from the difference impulse response of FIG. 8 constructed according to the principles of the present invention;

FIG. 9B illustrates an exemplary phase graph of the acoustic anti-transient-masking transform derived from the difference impulse response of FIG. 8 constructed according to the principles of the present invention;

FIG. 10 illustrates an embodiment of an acoustic anti-transient-masking transform system for compensating for effects of undesired vibrations impinging an audio component constructed according to the principles of the present invention; and

FIG. 11 illustrates a flow diagram of an embodiment of a method of compensating an audio signal for effects of undesired vibrations conducted in accordance with the principles of the present invention.

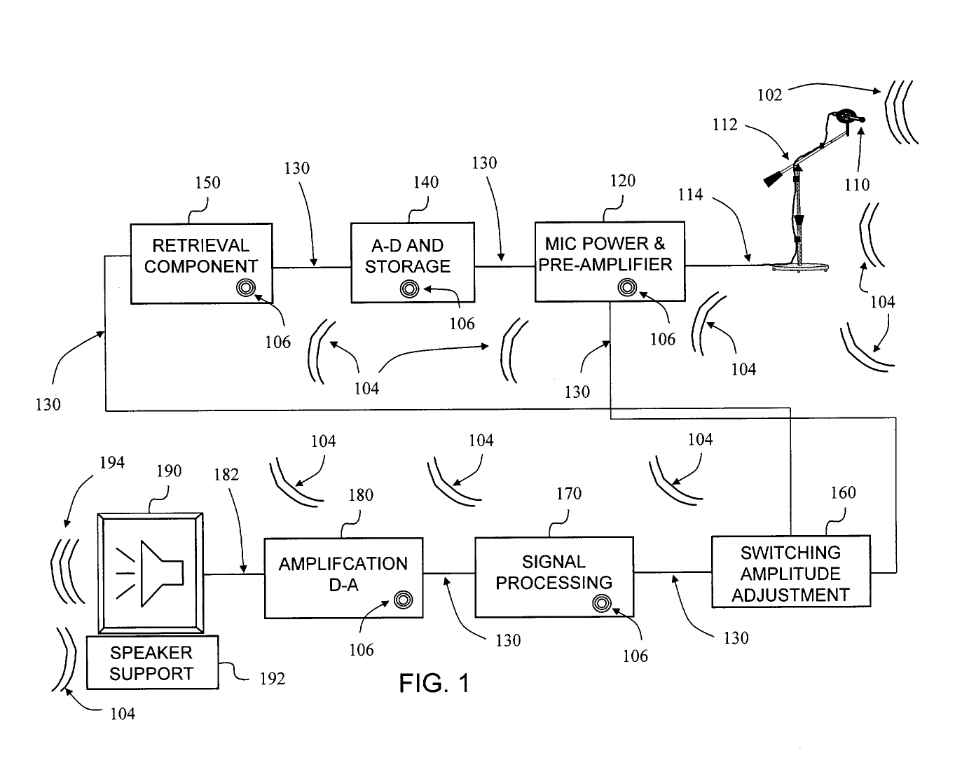

### DETAILED DESCRIPTION

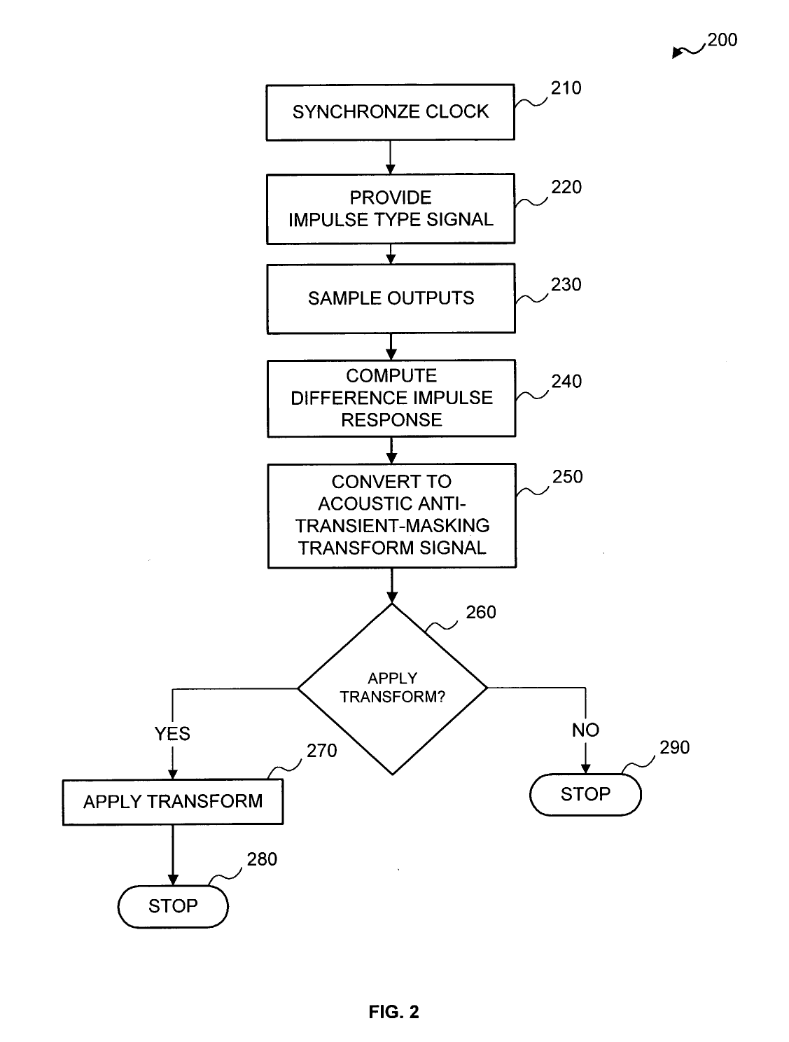

Referring now to FIG. 2, illustrated is a flow diagram of an embodiment of a method of developing an acoustic anti-transient-masking transform, generally designated 200, for compensating effects of undesired vibrations impinging on an audio component conducted in accordance with the principles of the present invention. The method 200 is operable on a variety of audio component. One audio component may be a microphone in a microphone holder. Another audio component may be a digital recording device the employs an analog-to-digital converter. Yet another audio component may be a digital playback device that employs a digital-to-analog converter. Still yet another audio component may be a receiver, an amplifier, an audio recording system or an equalizer. In yet another embodiment, the audio component may be a public address system. Of course, however, the present invention is not limited to the audio components listed above. In other embodiments of the present invention, the method may be employed with any type of audio component that is susceptible to the effect of undesired vibrations.

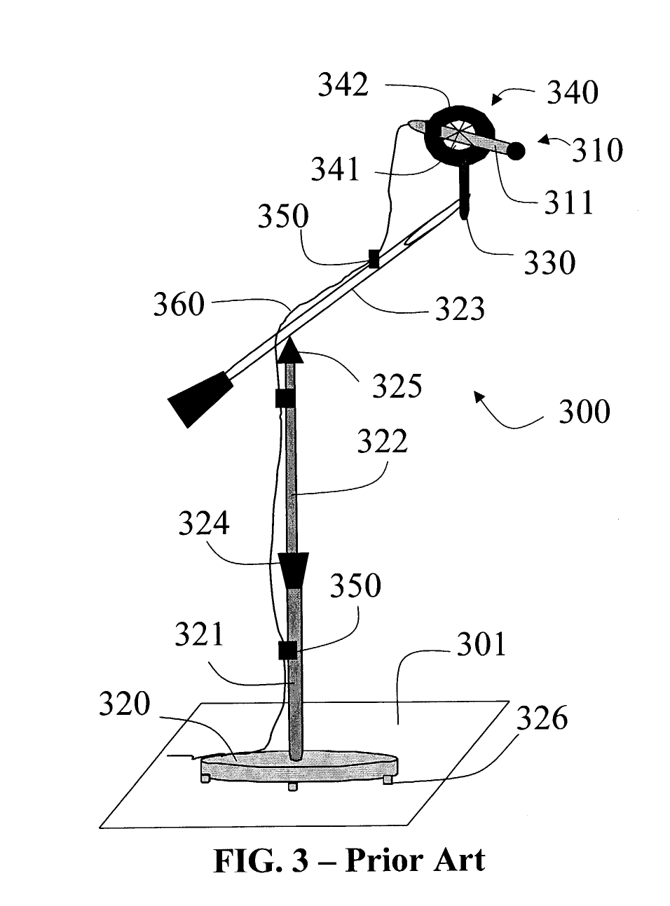

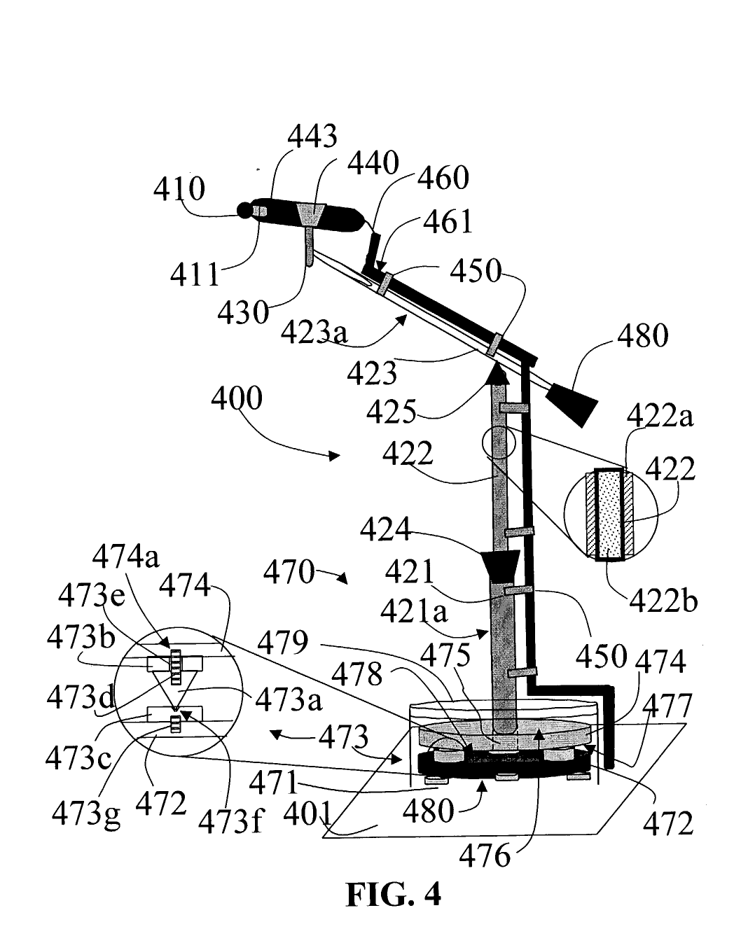

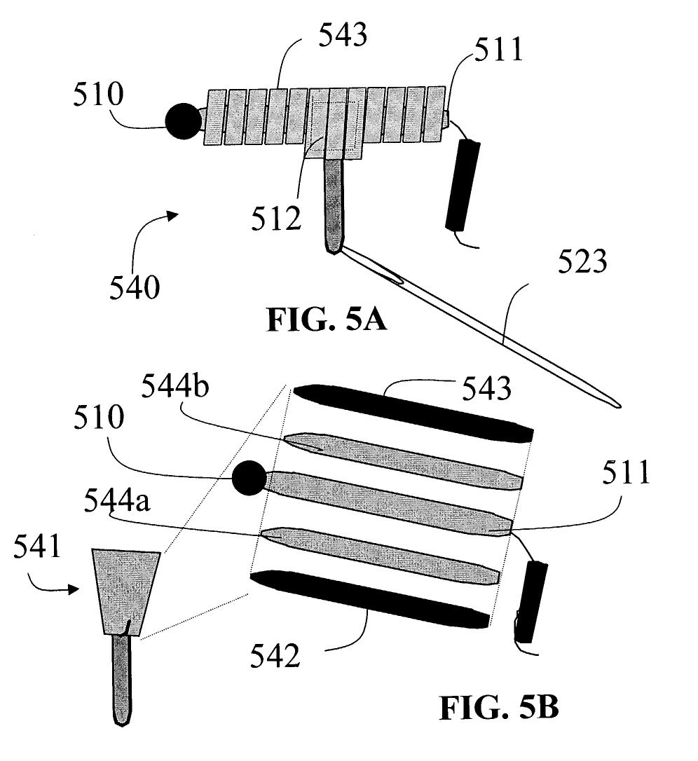

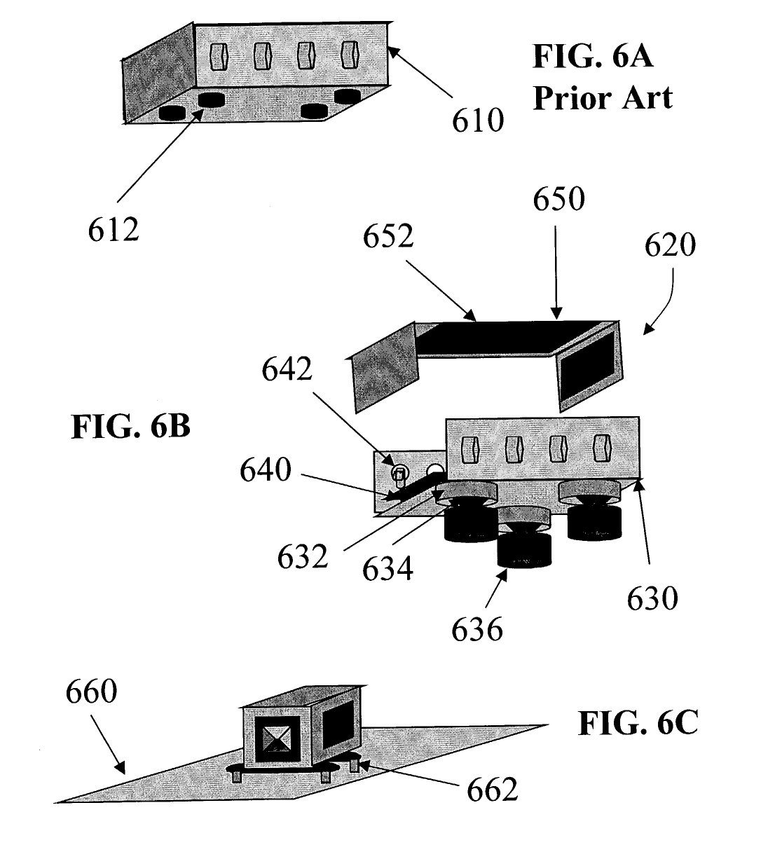

The method 200, in one embodiment, uses two versions of the audio component. The first version is an undesired vibration compensated version of the audio component. FIGS. 4, 5A and 5B illustrate and describe an exemplary undesired vibration compensated microphone support system. FIGS. 6B and 6C also illustrate and describe an exemplary undesired vibration compensated audio component other than a microphone support system. The second version is an uncompensated version of the audio component. FIG. 3 illustrates and describes a conventional microphone stand that is not compensated for undesired vibrations. Also, FIG. 6A illustrates and describes a conventional audio component that is not compensated for undesired vibrations.

If the audio component employs a clock circuit, the method 200 synchronizes the clocks between the two versions of the audio component in a step 210. In a related embodiment, the method 200 may employ a common clock signal between the compensated version and the uncompensated version of the audio component. The method 200 then provides an impulse type signal to the undesired compensated version and the uncompensated version in a step 220. For purposes of the present invention, the phrase “impulse type signal” means an imperfect impulse signal which is composed of substantially equal amounts of frequency content across a desired audio band for a particular audio component. For example, an exemplary impulse type signal may be one drum beat of a tympani drum. Of course, however, the present invention is not limited to only this one type of impulse type signal. Other embodiments of the present invention may employ other types of impulse type signals or particular types of impulse type signal for a given audio component.

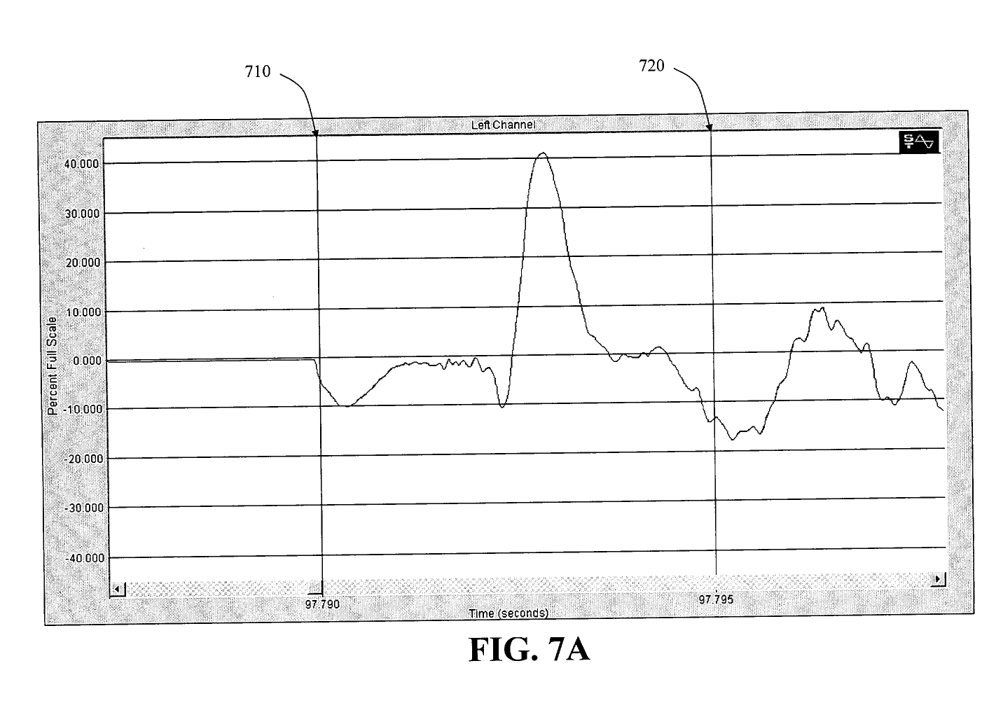

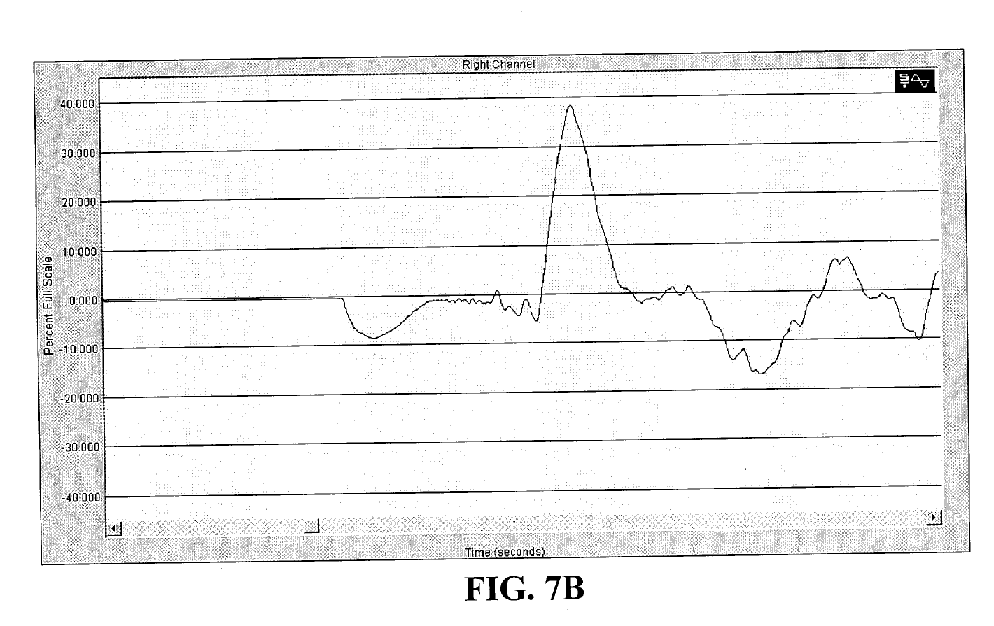

Next, the method 200 samples outputs from both of the compensated version and the uncompensated version in a step 230. FIG. 7A illustrates an exemplary time domain graph of the sampled output of the impulse type signal from the compensated microphone support system of FIG. 4. FIG. 7B illustrates an exemplary time domain graph of the sampled output of the same impulse type signal from an uncompensated version of a microphone stand similar to the one illustrated in FIG. 3. In a related embodiment, the method 200 substantially simultaneously samples the output of both the compensated version and the uncompensated version of the audio component.

In another embodiment, the method 200 may sample the outputs from the compensated version and the uncompensated version over a time period that includes substantially all of a response of the compensated version of the audio component and the uncompensated version of the audio component to the impulse type signal. Referring to FIG. 7A, a starting point of the time period of a response to the impulse type signal is illustrated by a line 710. This is typically when there is a relative amount of change in the energy of the response signal. An ending point of the time period of the response is illustrated by line 720. The ending point is typically at the point when response to the impulse type signal ends and noise starts. One skilled in the art should know that the time period that includes substantially all of a response depends upon the type of impulse type signal and the type of audio component.

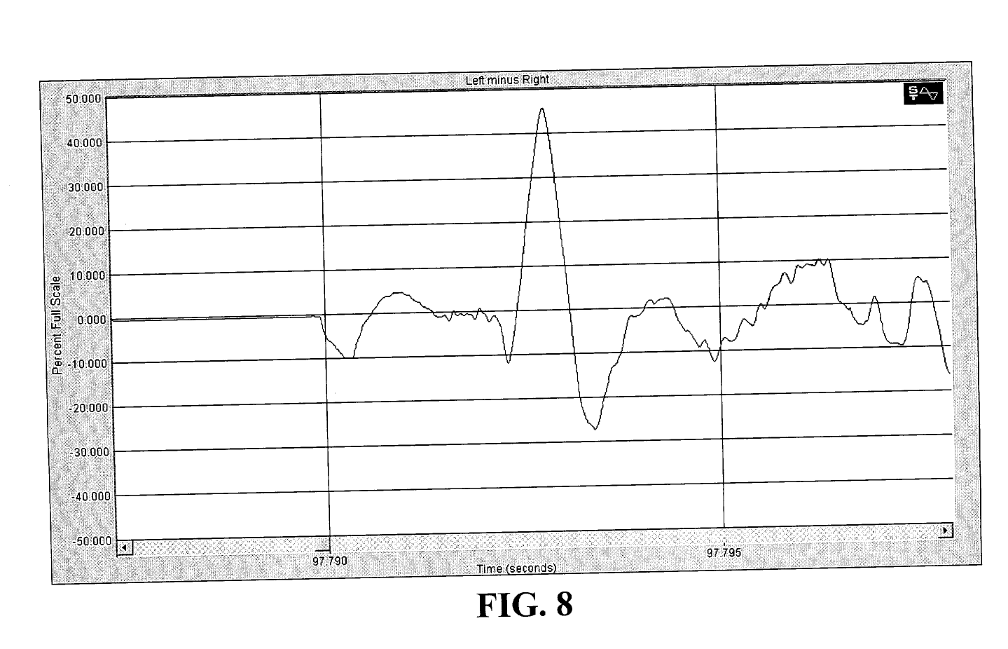

Referring back to FIG. 2, the method 200 then computes a difference impulse response between the sampled output from the compensated version and the sampled output from the uncompensated version in a step 240. In one embodiment, the method 200 computes the difference impulse response by subtracting the sampled output signal from the uncompensated version from the sampled output from the compensated version. FIG. 8 illustrates an exemplary time domain graph of the difference impulse response signal between the sampled output from the compensated version (FIG. 7A) and the sampled output from the uncompensated version (FIG. 7B). In another embodiment, the method 200 may compute the difference impulse response by subtracting the sampled output signal from compensated version from the sampled output from the uncompensated version.

Next, the method 200 converts the difference impulse response to a signal representing an acoustic anti-transient-masking transform in a step 250. For purposes of the present invention the phrase “acoustic anti-transient-masking transform” means a transform signal or transform signal representation that compensates for a misalignment of constituent frequency and/or phase components of an audio signal causing a degradation of sonic clarity and perceived and actual loss (masking) of transient responses.

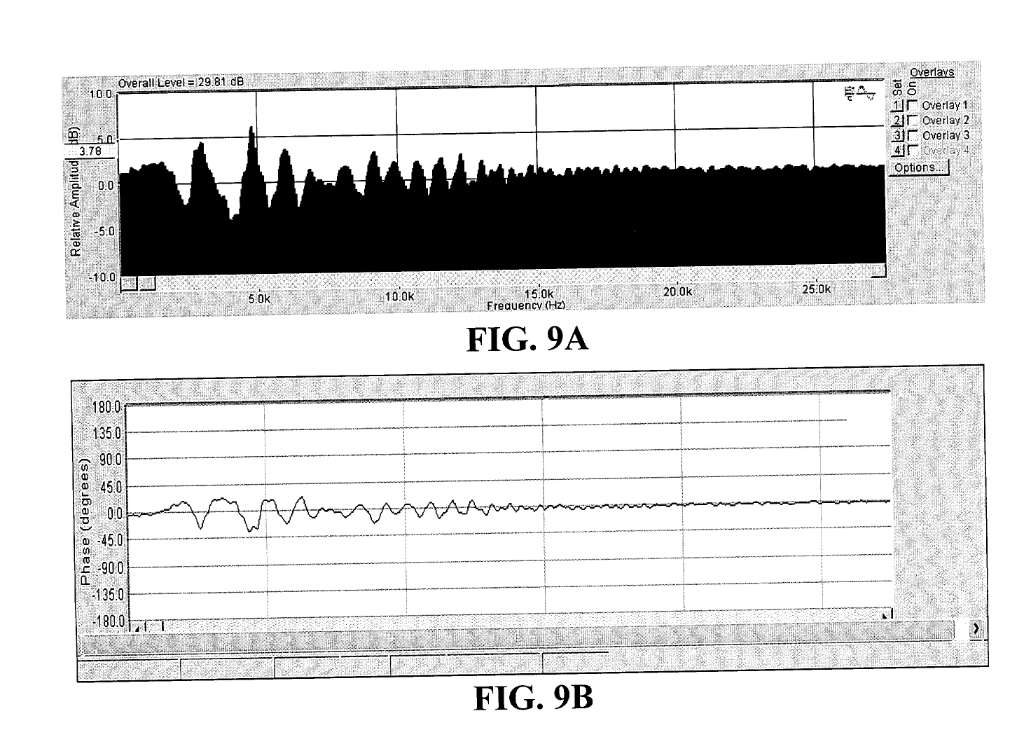

In one embodiment, the method 200 converts the difference impulse response (FIG. 8) by applying a Fast Fourier Transform (FFT) to the difference impulse response. FIG. 9A illustrates an exemplary magnitude graph of an acoustic anti-transient-masking transform derived from the difference impulse response of FIG. 8 constructed according to the principles of the present invention. FIG. 9B illustrates an exemplary phase graph of the acoustic anti-transient-masking transform derived from the difference impulse response of FIG. 8 constructed according to the principles of the present invention. One skilled in the art should know that the graphs illustrated in FIGS. 9A and 9B are only a portion of the entire acoustic anti-transient-masking transform signal. The graphs illustrate the low frequency range of the acoustic anti-transient-masking transform signal. Also, the present invention is not limited to sampling at a specific sampling rate. Other embodiments of the present invention may use different sampling rates and still be within the scope of the present invention. In a related embodiment, the method 200 may store the acoustic anti-transient-masking transform in a memory type device.

One skilled in the art should know that a time domain audio frequency signal can be thought of in terms of its constituent parts through the Fourier series theorem. Also, any signal can be described as consisting of, essentially, a sum of many different sinusoids, each with a strict relationship in gain and phase to one another, such that when combined they completely describe the desired complex signal. Consequently, when one or more of the constituent components of the Fourier series is (are) not maintained in the proper relationship with the others, transient response suffers since it is most susceptible to degradations where only a portion of the desired signal components maintain their integrity with respect to the others. When the proper balance of gain and phase of each of the component signals is altered, the subsequent recorded signal sounds “about right” but has lost the magic transient response that is perceived by human hearing as a real versus recorded/reproduced sound. This is one reason why the perceptual based coding schemes perform poorly in this area. These schemes remove essentially all of the frequency information above 7.7 KHz. Perceptual based coding schemes are based on the perceptual coding model of signal strength which asserts that human recognition of steady state sine wave signals above the 7.7 KHz frequency requires very large levels of amplitude to be perceived. The present invention claims that improved transient response and high sample rate/bit rate technology can provide increased levels of performance in recorded/reproduced audio signals and that there is another auditory mechanism in effect independent of high frequency tone (sine wave) response. This effect relates to the importance and presence of high frequency content in audio frequency sounds, somewhat independent of their steady state sine wave tone recognition, which provides valuable auditory cues with respect to transient response and clarity of sound. Additional background information concerning perceptual coding is discussed in “Perceptual Coding of Digital Audio,” by Ted Painter and Andreas Spanias, Proceedings of the IEEE, Volume 88, No. 4 (April 2000), which is hereby incorporated by reference in its entirety.

Referring back to FIG. 2, the method 200 then determines if the acoustic anti-transient-masking transform is to be applied to an audio stream in a decisional step 260. If the acoustic anti-transient-masking transform is to be applied, the method 200 multiplies the audio stream from an uncompensated audio component by the acoustic anti-transient-masking transform to compensate for effects of undesired vibrations impinging upon the uncompensated audio component in a step 270. Next, the method 200 stops in a step 280. If the method 200 determines that the acoustic anti-transient-masking transform is not be applied to an audio stream in the decisional step 260, the method 200 stops in a step 290.

One skilled in the art should know that the present invention is not limited to the described embodiment that includes computing a difference impulse response and then converting the difference impulse response to an acoustic anti-transient-masking transform. The present invention and method, in another embodiment, may employ an equivalent method that includes applying transforms, such as FFTS, to the sampled outputs from the compensated version and uncompensated version of the audio component and then computing a difference between the transformed signals to generate the signal that represents the acoustic anti-transient-masking transform. Also, other embodiments of the present invention may have additional or fewer steps than described above.

Referring now to FIG. 3, illustrated is conventional microphone stand, generally designated 300, uncompensated for undesired vibrations. The conventional microphone stand 300 comprises a base 320, a first vertical support pole 321, a second vertical support pole 322, an adjustable support pole 323, a first support pole clutch assembly 324, a second support pole clutch assembly 325, a pole-to-microphone adapter 330, a microphone holder 340, and cable clamps 350. The microphone stand 300 stands upon a floor 301 and supports a conventional microphone 310. The microphone 310 has a microphone body 311 coupled to a microphone cable 360. The microphone cable 360 is coupled to the first vertical support pole 321, the second vertical support pole 322, and the adjustable support pole 323 with the cable clamps 350. In the embodiment shown, the base 320, the first vertical support pole 321, second vertical support pole 322, adjustable support pole 323, first support pole clutch assembly 324, second support pole clutch assembly 325, pole-to-microphone adapter 330, microphone holder 340, and cable clamps 350 typically comprise resonant materials such as metal, hard plastic, etc. In one embodiment, the base 320 may have rubber feet 326 to decouple vibration arising from the floor 301.

The major effect of the various vibration sources is the microphonic transfer of vibration to the microphone electronics through the microphone stand/holder assembly and microphone wiring. The vibrations subsequently modulate the desired program material as a by-product of sensing and amplification. In most cases little special care has been taken to isolate the microphone sensing element(s) (not shown) from the microphone body 311. In an embodiment considered to be among the best of the prior art, the microphone holder 340 comprises some form of elastic suspension bands 341 coupled between a circumferential ring 342 and the microphone 310. Various forms of this general method of isolation are disclosed in U.S. Pat. No. 6,459,802 to Young, U.S. Pat. No. 4,546,950 to Cech, U.S. Pat. No. 4,396,807 to Brewer, U.S. Pat. No. 4,194,096 to Ramsey, ostensibly to isolate the microphone 310 from floor-borne, low frequency vibrations. The above listed patents are hereby incorporated by reference. While it is desirable to isolate the microphone/stand combination from floor-borne vibrations, the methods of the prior art subject the microphone elements to significantly larger degradations from airborne vibrations through the microphone enclosure (the microphone body 311 or case) which is generally not protected in any way from airborne vibrations. Undesired vibrations can be additionally magnified when the microphone (sensor) is suspended via these weblike mechanisms, as in the listed prior art, in an effort to isolate it from the low frequency vibrations transmitted from the floor. This is accomplished at the expense of exposure to the significantly higher levels and wider frequency spectrum of vibration levels available directly through the air. These vibrations must also be addressed in the quest to control the recording/reproduction process in an effort to preserve the transient response of the desired signal to be recorded or processed. With the prior art, the conventional microphone 310 receives, and inadvertently converts to an electrical signal, those vibrations it receives through the microphone body 311 and the microphone cable 360, along with the airborne vibrations sensed by the microphone element from the desired signal. Vibrations in the microphone stand/holder assembly also can cause very small movements of the entire microphone 310, and therefore the element(s) of the microphone while it is receiving the desired signal. Vibrations of the microphone stand 300 also cause a lever arm effect on the suspended microphone 310 which magnifies the effect of small vibrations in the microphone stand 300.

In most cases little special care has been taken to isolate the microphone sensing element(s) from the microphone body 311. Generally, the microphone 310 itself is, in the presumed best form of the prior art, suspended in air via elastic suspension bands 341, ostensibly to isolate it from floor-borne low frequency vibrations. While it is desirable to isolate the microphone 300 and the microphone stand 300 from floor-borne vibrations, the method of the prior art subjects the microphone assembly to significantly larger degradations from airborne vibrations through its enclosure (the microphone body 311) which is not protected in any way from undesired airborne vibrations. Ideally, the best mounting mechanism would reveal the main (desired) sensing element(s) to the sounds to be converted into electrical signals, while keeping the body of the microphone 310, and therefore the remaining electronics inside it, isolated from undesired airborne vibrations. With the prior art, the microphone 310 receives and inadvertently converts vibrations it receives through its body 311 and the microphone wire 360, along with the vibrations sensed by the main (desired) element from the desired signal. Consequently, any vibrations, including undesired solid-body vibrations, received through the microphone body 311 or its holding mechanism 340, stand 300, and cabling 360 get combined with the desirable sounds from an intended source impinging on the main microphone element(s); thereby the net combination of these signals becomes the overall signal produced by the microphone 310, microphone holding system 300, and cabling 360.

Referring now to FIG. 4, illustrated is one embodiment of a microphone support system, generally designated 400, compensated for undesired vibrations constructed according to the principles of the present invention. In the illustrated embodiment, the microphone support system 400 comprises a first vertical support pole 421, a second vertical support pole 422, an adjustable support pole 423, a first support pole vibration-conducting coupling 424, a second support pole vibration-conducting coupling 425, a pole-to-microphone adapter 430, a microphone holder 440, a microphone sheath 443, cable clamps 450, a base assembly 470, and a counterweight 480. The microphone support system 400 supports a conventional microphone 410 that has a microphone body 411. The microphone body 411 is electrically and mechanically coupled to a microphone cable 460. In a preferred embodiment, the microphone cable 460 is substantially surrounded about its entire length with a vibration-absorbing coating 461 that substantially isolates the microphone 410 from at least some of any vibration that might impinge on the microphone cable 460. In one embodiment, only those areas of the microphone cable 460 very close to the microphone body 411, and to the recording/reproduction electronics (not shown) are not covered with the vibration-absorbing coating/sheath 461. In a preferred embodiment, the vibration-absorbing coating/sheath 461 is polystyrene foam. The microphone cable 460 is mechanically coupled to the first vertical support pole 421, the second vertical support pole 422, and the adjustable support pole 423 with the cable clamps 450. In the illustrated embodiment, the microphone support system 400 is designed to be placed on a support element 401 that may be subjected to undesired vibrations. In the embodiment shown, the support element 401 is a conventional floor, presumably of a musical performance/recording studio, although the microphone support system 400 may be used at other locations, e.g. a stage, meeting room, etc. In another embodiment, the support element may be a desk (not shown) or any surface suitably strong enough to support the microphone support system 400. In such a desk-mounted system, as one who is skilled in the art will readily understand, the size and-number of the support poles may be significantly reduced while the general principles of the present invention are applied. The undesired vibrations may be caused by any of the previously listed sources including, but not limited to: a live music source, e.g., musical instruments, and the heating ventilation and air conditioning system (HVAC), etc.

Details of two embodiments of the microphone holder will be addressed below with reference to FIGS. 5A and 5B. For the sake of the present discussion, it is sufficient to note that the conventional microphone 410 is substantially surrounded by vibration-absorbing or vibration-resistant material (microphone sheath 443) in accordance with the principles of the present invention.

In one embodiment, the base assembly 470 comprises vibration-isolating feet 471, a vibration-resistant sub-base 472, vibration-absorbing receptacles 473, a non-resonant base 474, and a base assembly cover 479. In a preferred embodiment, the non-resonant base 474 comprises a circular base made of carbon fiber material such as is produced by Black Diamond Racing, Inc. (BDR), a division of D. J. Casser Enterprises, Inc., Milwaukee, Wis. In one embodiment, the diameter of the non-resonant base 474 may be between about 16″ and 18″. In a preferred embodiment, the non-resonant base 474 may have a threaded hole 475 for coupling to the first vertical support pole 421. In another embodiment, an upper surface 476 of the non-resonant base 474 may have a threaded flange (not shown) coupled to it for coupling to the first vertical support pole 421. One who is skilled in the art is familiar with the use of threaded flanges for coupling threaded poles to flat surfaces. Performance of the recording/reproduction system was noticeably better with the threaded hole 475 embodiment.

In one embodiment, the vibration-absorbing receptacles 473 may comprise carbon fiber “cones” 473a, “pucks” 473b, and “pits” 473c. The cones 473a, pucks 473b and pits 473c may be ones available from BDR. The cones 473a comprise solid carbon fiber formed as a cone with an imbedded threaded rod 473d. In a preferred embodiment, the non-resonant base 474 may have a plurality of threaded holes 474a in a lower surface 477 thereof to which the cones 473a and pucks 473b may be coupled in a point-down configuration. The pucks 473b also comprise carbon fiber similar in appearance to a hockey puck with a central hole 473e. The pits 473c are coupled to an upper surface 478 of the sub-base 472 and have a depression 473f on one surface that receives the point of a cone 473a. In the illustrated embodiment, the pits 473c may include an imbedded threaded rod 473g used to coupled the pits 473c to the upper surface 478 of the sub-base 472. In a preferred embodiment, at least three pairs of pucks 473b, cones 473a, and pits 473c are employed.

In a preferred embodiment, the vibration-resistant sub-base 472 comprises a circular oak plywood disk of a similar size to the non-resonant base 474. In one embodiment, the sub-base 472 is 1.25 inch thick, circular oak plywood that is a substantially non-resonant material. In one embodiment, the sub-base 472 may additionally be coated with an additional, non-resonant material, such as a fiberglass-reinforced epoxy resin, to further reduce susceptibility to vibration. A suitable fiberglass-reinforced polyester/epoxy resin is Evercoat®, a product of the Fibre Glass-Evercoat Company of Cincinnati, Ohio. In one embodiment, an upper surface 478 of the sub-base 472 may have threaded holes (not shown) configured to accept mounting bolts for BDR “Thick Pits.” The Thick Pits have deep dimples 473f on their exposed surface to receive points of the cones 473a. The vibration-resistant sub-base 472 absorbs, through the vibration-absorbing receptacles 473, at least some of the vibration that may impinge upon the entire microphone support system 400.

In a preferred embodiment, the sub-base 472 has vibration-isolating feet 471 coupled to an undersurface 480 of the sub-base 472. The vibration-isolating feet 471 serve to substantially isolate the vibration-resistant sub-base 472 from at least some of the floor-borne vibrations. In a preferred embodiment, the vibration-isolating feet 471 may comprise rubber bushings. In another embodiment, the rubber bushings may be a type 6 (ribbed bushing) or type 7 (ribbed ring) commonly available from the McMaster-Carr Company of Atlanta, Ga.

The base assembly 470 may further comprise a base assembly cover 479 substantially surrounding the sub-base 472, the vibration-isolating feet 471 and the non-resonant base 474. The base assembly cover 479 couples to the base assembly 470 by surrounding the first vertical support pole 421 and substantially shields the base assembly 470 from at least some of any undesired vibrations, including airborne vibrations. The vibration-isolating feet 471 substantially isolate the sub-base 472 from floor-borne vibrations.

The base assembly 470 is coupled to the first vertical support pole 421 as detailed above with or without a flange. In turn, the first vertical support pole 421 is coupled to the second vertical support pole 422 with the first support pole vibration-conducting coupling 424. The second vertical support pole 422 is coupled to the adjustable support pole 423 with the second support pole vibration-conducting coupling 425. In a preferred embodiment, the first and second support pole vibration-conducting couplings 424, 425 are constructed of substantially non-resonant material such as a brass collet and a brass jamb nut. However, these first and second support pole vibration-conducting couplings 424, 425 are vibration conducting, and will serve to conduct any vibrations impinging upon the microphone body 411 down into the base assembly 470.

Additionally, the first vertical support pole 421, second vertical support pole 422 and the adjustable support pole 423 may be surrounded or coated with a vibration-damping coating 421a, 422a, 423a. The vibration-damping coating may be a flexible rubber. Suitable flexible rubber coatings are also available from McMaster-Carr. In another embodiment, the vibration-damping coating may be polystyrene foam. In yet another embodiment, the vibration-damping coating may be polyethylene foam. In still yet another embodiment, the vibration-damping coating may be elastomeric foam. In a similar manner, the first support pole vibration-conducting coupling 424 and the second support pole vibration-conducting coupling 425 may be constructed of brass, which is substantially non-resonant. In this embodiment, the second vertical support pole 422 and the adjustable support pole 423 may be advantageously hollow and therefore filled with a vibration-damping filler 422b to effectively dampen the normal resonant modes of the support poles 422, 423 while allowing high frequency vibrations to be transmitted to the absorbing base assembly 470. In one embodiment, the vibration-damping filler 422b comprises lead and sand. In a preferred embodiment, the vibration-damping filler 422b is a 50/50 mixture by volume of #7 or #8 lead shot and play sand.

Referring now to FIG. 5A with continuing reference to FIG. 4, illustrated is one embodiment of a microphone holder, generally designated 540, compensated for undesired vibrations constructed according to the principles of the present invention. In the illustrated embodiment, a conventional microphone 510 has a microphone body 511 and a hard mount 512 for coupling to a conventional microphone stand 523. The hard mount 512 also provides for the vibration coupling of the microphone body 511 to the microphone stand 400 of FIG. 4. In this embodiment, the microphone holder 540 comprises a microphone sheath 543 of vibration-absorbing material substantially isolating the microphone 510 from at least some of any undesired vibration. In one embodiment, the vibration-absorbing material is foam rubber. In another embodiment, the vibration-absorbing material is a polymer resin. In a perferred embodiment, the vibration-absorbing material is Rubatex insulation tape. Rubatex insulation tape is a closed cell, polymer foam insulation tape manufactured by RBX Industries, Inc., of Roanoke, Va. The insulation tape may be wrapped and shaped to ensure minimal impact on the reception pattern of the microphone 510 as well as thorough coverage of the exposed microphone body 511. The use of vibration-absorbing material allows the sheath 543 to absorb undesired vibrations, such as airborne vibrations, prior to the vibration's impact on the microphone body 511. The result is that the microphone 510 is shielded from undesired vibration, and whatever vibration the microphone body 511 does receive is channeled downward through the stand 400 into the base assembly 470 where absorbing material dissipates the vibration.

Referring now to FIG. 5B, illustrated is an alternative embodiment of a microphone holder 541 compensated for undesired vibrations constructed according to the principles of the present invention. In the illustrated embodiment, the conventional microphone 510 has a microphone body 511 but does not have a hard mount for coupling to a conventional microphone stand, thereby requiring a different approach. A microphone 510 of this type typically uses a holder shaped like a circle, or semi-circle, into which the microphone 510 is slid, or a clamp of some sort to grab the microphone body 511 in order to hold the microphone 510. In this embodiment, the microphone holder 541 comprises a two-part outer shell 542, 543, and an inner packing 544 shown as two parts 544a, 544b. In one embodiment, the two-part outer shell 542, 543 comprises a section of PVC pipe shorter than the length of the microphone 510 and cut lengthwise to create two halves 542, 543. The two halves 542, 543 have rounded/sculpted ends to minimize the shielding effect on the desired reception pattern of the basic microphone 510. In a preferred embodiment, the inner packing 544 comprises a lining of the two halves 542, 543 with Evercoat. The Evercoat lining comprises a densely packed fiberglass material which allows a good vibration-resistive coupling to the microphone body 511 while enabling a channeling of vibration received by the PVC halves 542, 543 down into the microphone stand. This effectively isolates the microphone 510 from both airborne and floor-borne vibrations. It should be understood that the alternative microphone holder embodiments of FIGS. 5A and 5B may be employed with any of the microphone stand, such as the embodiment illustrated in FIG. 4.

Referring now to FIG. 6A, illustrated is an uncompensated audio component 610. The uncompensated audio component 610 may be any type of audio component having a chassis and electronic circuitry. The uncompensated audio component 610 may include conventional feet 612. The feet 612 may be composed of rubber. However, the audio component 610 is not compensated for undesired vibrations as the audio component. For a discussion on compensating for undesired vibrations see FIGS. 6B and 6C.

FIG. 6B illustrated is an audio component, generally designated 620, compensated for undesired vibrations constructed according to the principles of the present invention. The compensated audio component 620 includes a chassis 630 that houses the electronic components, circuit boards, switches, connections of the audio component 620. Coupled to the bottom of the chassis are vibration resistant pucks 632. Coupled to the pucks 632 are vibration resistant cones 634 and coupled to the cones 634 are vibration resistant pits 636. The pucks 632, cones 634 and pits 636 may be made of carbon fiber, and may be the similar types of pucks, cones and pits of FIG. 4.

In the illustrated embodiment, the compensated audio component 620 includes a circuit board 640 that is “shock mounted” to the chassis 630 employing isolation techniques to limit vibration transfer from the chassis to the circuit board 640. If the compensated audio component 620 includes additional circuit boards 640, the additional circuit boards are also shock mounted. Also, if the circuit board includes one or more crystals (not shown), such as a clock reference, the crystals should be mounted in such a way that the crystals' axis of most sensitivity is exposed to the audio component's axis of least vibration. One skilled in the art knows that crystal structures and implementations have different directional sensitivities, that is, a crystal may be more sensitive in one axis than another axis.

The compensated audio component 620 may also include connectors 642 that are used for passing signals in and out of the audio component. The connectors 642 are mounted to the circuit board 640 only and are not mounted to or attached to the chassis 630. The chassis 630 also employs open holes in the front and rear of the chassis 630 to allow the connectors 642 and any controls to protrude for easy connection and/or operation. This aids in the reducing the undesired vibrations from being transmitted (impinged) through the chassis 630 to the connectors 640 and controls to the circuit board 640. In addition, the compensated audio component 620 includes a cover 650 having vibration shielding material 652 on the inside of the cover 650. The vibration shielding material 652 may also be on the inside of the chassis 630. The vibration shielding material 652 may be the same or similar material as Evercoat, described previously.

Referring now to FIG. 6C, illustrated is an exemplary chassis bottom plate 660 of the compensated audio component 620. If the compensated audio component 620 includes a transformer 662, the transformer 662 is “shock mounted” to the chassis bottom plate 660 employing isolation techniques to limit vibration transfer from the transformer to the chassis 630 and any circuit boards 640. In one embodiment, the transformer 662 may employ isolation grommets instead of direct mount using screws or bolts. In another embodiment, the transformer 662 may employ floating mounts that lock for shipment but release for use (may twist the mounting mechanism to release). Of course, however, one skilled in the art should know that the different types of audio components require different types of compensation to compensate for undesired external and internal vibrations.

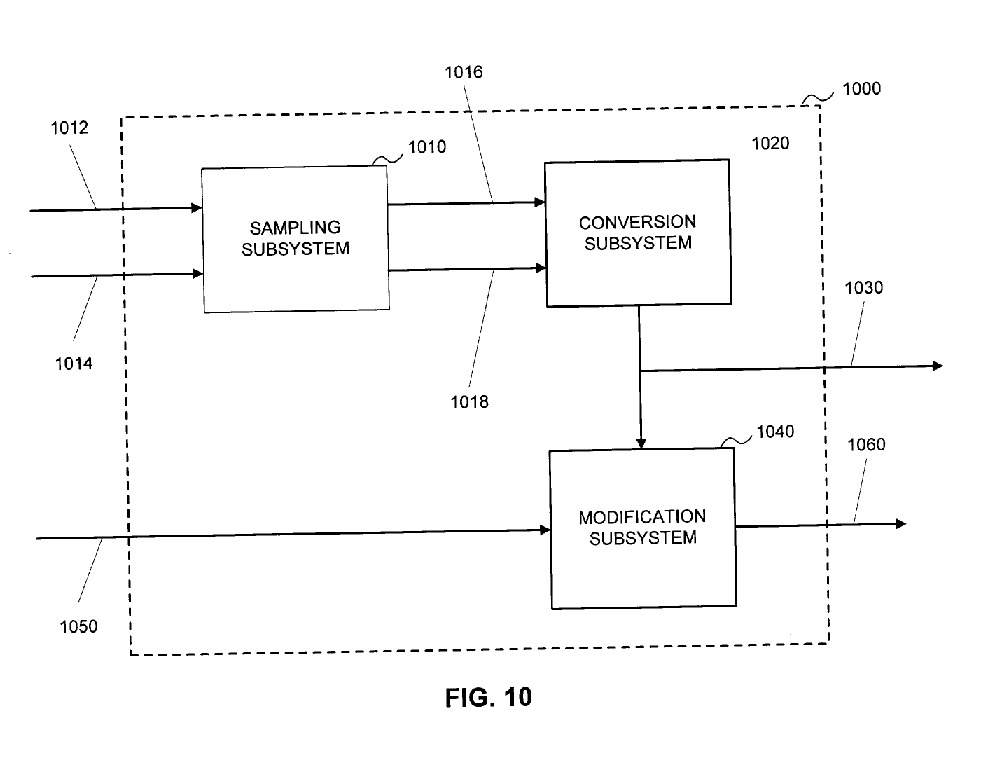

Referring now to FIG. 10, illustrated is an embodiment of an acoustic anti-transient-masking transform system, generally designated 1000, for compensating for effects of undesired vibrations impinging an audio component constructed according to the principles of the present invention. The acoustic anti-transient-masking transform system 1000 may be embodied in hardware, software, firmware or a combination thereof. In another embodiment, the acoustic anti-transient-masking transform system 1000 may be embodied in a digital signal processor, a fast programable gate array, an application specific integrated circuit, a special purpose FFT integrated circuit, or a digital editing workstation. The acoustic anti-transient-masking transform system 1000 includes a sampling subsystem 1010 and a conversion subsystem 1020.

The sampling subsystem 1010 is configured to digitally sample a first output 1012 from an undesired vibration compensated version of the audio component and produce a first sampled output 1016. The first output 1012 is based upon a response to an impulse type signal supplied to the compensated version of the audio component associated with the first output 1012. See FIGS. 4 and 6B for examples of undesired vibration compensated audio components. The sampling subsystem 1010 is further configured to digitally sample a second output 1014 from an uncompensated version of the audio component and produce a second sampled output 1018. The second output 1014 is based upon a response to an impulse type signal supplied to the uncompensated version of the audio component associated with the second output 1014. See FIGS. 3 and 6A for examples of uncompensated audio components. In another embodiment, the sampling subsystem 1010 may digitally sample the first and second outputs 1012, 1014 substantially simultaneously. Also, if the compensated version and the uncompensated version of the audio component includes a clock circuit, then the uncompensated version and the compensated version may be configured to use a common clock signal. In yet another embodiment, the sampling subsystem 1010 may be embodied in an analog-to-digital converter.

In addition, the sampling subsystem 1010 may sample the first and second outputs 1012, 1014 over a time period that includes substantially all of a response of the compensated version of the audio component and the uncompensated version of the audio component to the impulse type signal. FIG. 7A illustrates and exemplary a starting point 710 of the time period of a response to the impulse type signal. This is typically when there is a relative amount of change in the energy of the response signal. An ending point 720 of the time period of the response is typically at the point when response to the impulse type signal ends and noise starts.

The conversion subsystem 1020 is configured to receive the first and second sampled outputs 1016, 1018 and compute a difference impulse response between the first and second sampled outputs 1016, 1018. In one embodiment, the conversion subsystem 1020 may compute the difference impulse response by subtracting the second sampled output 1018 from the first sampled output 1016. Of course, however, the difference impulse response could also be determined by subtracting the first sampled output 1016 from the second sampled output 1018. The conversion subsystem 1020 is further configured to convert the difference impulse response to a signal 1030 representing an acoustic anti-transient-masking transform. In another embodiment, the conversion subsystem 1020 is further configured to convert the difference impulse response by applying a FFT to the difference impulse response.

In an alternative embodiment, the conversion subsystem 1020 is configured to apply a transform to the first and second sampled outputs 1016, 1018. The transform, one embodiment, may be a FFT. The conversion subsystem 120 is further configured to compute the difference between the two transformed signals to generate a signal 1030 representing the acoustic anti-transient-masking transform.

In the illustrated embodiment, the acoustic anti-transient-masking transform system further includes a modification subsystem 1040. The modification subsystem 1040 is configured to receive an audio stream 1050 (or signal) from an uncompensated audio component and the signal 1030 representing an acoustic anti-transient-masking transform. In one embodiment, the signal 1030 may be a digital representation of the acoustic anti-transient-masking transform or a portion thereof. The modification subsystem 1040 is further configured to multiply the audio stream 1050 by the signal 1030 representing the acoustic anti-transient-masking transform to generate an audio output 1060 that is compensated for the effects of undesired vibrations impinging the uncompensated audio component.

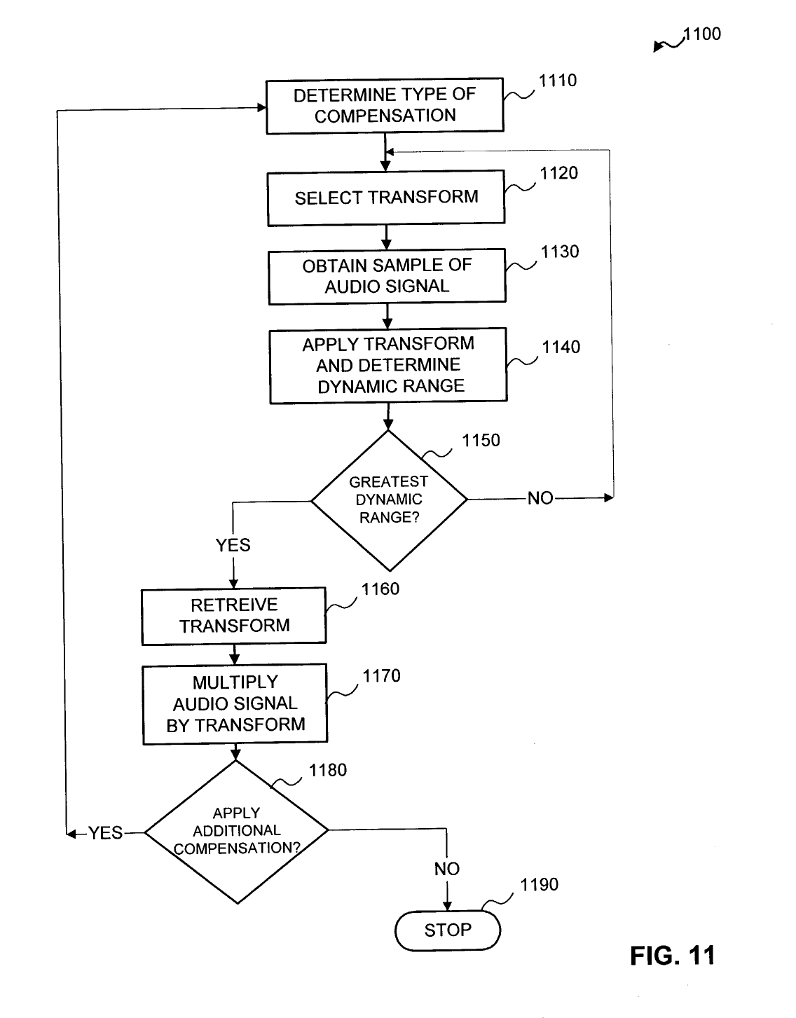

Referring now to FIG. 11, illustrated is a flow diagram of an embodiment of a method of compensating an audio signal, generally designated 1100, for effects of undesired vibrations conducted in accordance with the principles of the present invention. The method 1100 starts by determining a type of undesired vibration compensation to apply to the audio signal in a step 1110. The undesired vibration compensation may be an acoustic anti-transient-masking transform of FIGS. 2 and 10. One type of undesired vibration compensation may be a recording undesired vibration compensation. A recording undesired vibration compensation is an acoustic anti-transient-masking transform that was created for compensation during an audio recording process. Another type of undesired vibration compensation may be a post-record undesired vibration compensation, which compensates during the playback of the audio for undesired vibrations that occurred when the audio was recorded. Yet another type of undesired vibration compensation is an audio component undesired vibration compensation, which compensated for undesired vibrations of the audio component playing or reproducing the audio. Still yet another type of undesired vibration compensation is a sound amplification undesired vibration compensation, which compensated for undesired vibrations during amplification. For example, a live concert or a public address system. One skilled in the art should know that each type of undesired vibration compensation may also be dependent upon the type of audio component(s) employed.

Next, the method 1100, as part of determining the type of undesired compensation, may select an acoustic anti-transient-masking transform from multiple in a step 1120. The selected acoustic anti-transient-masking transform may be selected from multiple acoustic anti-transient-masking transforms having various types of undesired vibration compensations. The method 1100 may then obtain a sample of an audio signal in a step 1130. Employing this sample, the method 1100 may then apply the selected acoustic anti-transient-masking transform and determines a dynamic range for the sample in a step 1140.

The method 1100 then determines if the applied acoustic anti-transient-masking transform produced the greatest dynamic range in a decisional step 1150. If it did not produce the greatest dynamic range, then the method 1100 returns to select another acoustic anti-transient-masking transform in the step 1120. If the applied acoustic anti-transient-masking transform did produce the greatest dynamic range, the method 1100 then retrieves the appropriate acoustic anti-transient-masking transform in a step 1160. Next, the method 1100 multiplies the audio signal by the retrieved acoustic anti-transient-masking transform to generate an output signal compensated for the effects of the undesired vibrations in a step 1170. In a related embodiment, the method 1100 may employ an amplitude offset as part of the multiplication process to adjust the acoustic anti-transient-masking transform. In another embodiment, the method 1100 may employ a phase offset as part of the multiplication process to adjust the acoustic anti-transient-masking transform. In yet another embodiment, the method 1100 may employ a null spacing percentage as part of the multiplication process to adjust the acoustic anti-transient-masking transform.

The method 1100 may then determine if any additional undesired vibration compensations are to be applied in a decisional step 1180. If no additional compensation is required, the method 1100 stops in a step 1190. If additional compensation is required, the method 1100 returns to determine the next type of undesired vibration compensation to apply in the step 1110. One skilled in the art should know that other embodiments of the present invention may have additional or fewer steps than described above.

While the methods disclosed herein have been described and shown with reference to particular steps performed in a particular order, it will be understood that these steps may be combined, subdivided, or reordered to form an equivalent method without departing from the teachings of the present invention. Accordingly, unless specifically indicated herein, the order and/or the grouping of the steps are not limitations of the present invention.

Although the present invention has been described in detail, those skilled in the art should understand that they can make various changes, substitutions and alterations herein without departing from the spirit and scope of the invention in its broadest form.

What is claimed is:

1. A method of developing an acoustic anti-transient-masking transform for compensating effects of undesired vibrations impinging an audio component, comprising:
providing an impulse type signal to an undesired vibration compensated version of said audio component and an uncompensated version of said audio component;
computing a difference impulse response between a first sampled output from said compensated version and a second sampled output from said uncompensated version; and
converting said difference impulse response to a signal representing said acoustic anti-transient-masking transform.

2. The method as recited in claim 1 wherein said first and second sampled outputs are obtained by substantially simultaneously sampling of both of the outputs from said compensated version and said uncompensated version.

3. The method as recited in claim 1 wherein said first and second outputs are sampled over a time period that includes substantially all of a response of said compensated version and said uncompensated version to said impulse type signal.

4. The method as recited in claim 1 wherein said computing said difference impulse response includes employing a common clock signal if said audio component employs a clock circuit.

5. The method as recited in claim 1 wherein said computing a difference impulse response includes subtracting said second sampled output from said first sampled output.

6. The method as recited in claim 1 wherein said converting said difference impulse response includes applying a Fast Fourier Transform to said difference impulse response.

7. The method as recited in claim 1 further comprising multiplying an audio stream from said uncompensated audio component by said acoustic anti-transient-masking transform to compensate for effects of said undesired vibrations impinging said uncompensated audio component.

8. The method as recited in claim 1 wherein said audio component is selected from the group consisting of:
a microphone in a microphone holder,
a digital recording device employing an analog-to-digital converter,
a digital playback device employing a digital-to-analog converter,
a receiver,
an amplifier,
an audio recording system,
an equalizer, and
a public address system.

9. An acoustic anti-transient-masking transform system for compensating effects of undesired vibrations impinging an audio component, comprising:
a sampling subsystem configured to digitally sample a first output from an undesired vibration compensated version of said audio component in response to an impulse type signal and generate a first sampled output therefrom, said sampling subsystem further configured to digitally sample a second output from an uncompensated version of said audio component in response to said impulse type signal and generate a second sampled output therefrom;
a conversion subsystem configured to compute a difference impulse response between said first sampled output and second sampled output, and covert said difference impulse response to a signal representing an acoustic anti-transient-masking transform.

10. The acoustic anti-transient-masking transform system as recited in claim 9 wherein said sampling subsystem is further configured to digitally sample said first and second outputs substantially simultaneously.

11. The acoustic anti-transient-masking transform system as recited in claim 9 wherein said sampling subsystem is further configured to digitally sample said first and second outputs over a time period that includes substantially all of a response of said compensated version and said uncompensated version to said impulse type signal.

12. The acoustic anti-transient-masking transform system as recited in claim 9 wherein said compensated version and said uncompensated version are configured to use a common clock signal if said audio component employs a clock circuit.

13. The acoustic anti-transient-masking transform system as recited in claim 9 wherein said conversion subsystem is further configured to compute said difference impulse response by subtracting said second sampled output from said first sampled output.

14. The acoustic anti-transient-masking transform system as recited in claim 9 wherein said conversion subsystem is further configured to convert said difference impulse response by applying a Fast Fourier Transform to said difference impulse response.

15. The acoustic anti-transient-masking transform system as recited in claim 9 further comprising a modification subsystem configured to multiply an audio stream from said uncompensated audio component by said acoustic anti-transient-masking transform to compensate for effects of said undesired vibrations impinging said uncompensated audio component.

16. The acoustic anti-transient-masking transform system as recited in claim 9 wherein said audio component is selected from the group consisting of:
a microphone in a microphone holder,
a digital recording device employing an analog-to-digital converter,
a digital playback device employing a digital-to-analog converter,
a receiver,
an amplifier,
an audio recording system,
an equalizer, and
a public address system.

17. The acoustic anti-transient-masking transform system as recited in claim 9 wherein said conversion subsystem is configured to apply a transform to said first and second sampled outputs to produce a first and second transformed signals, and compute a difference between said first and second transformed signals to generate said signal representing said acoustic anti-transient-masking transform.

18. A method of compensating an audio signal for effects of undesired vibrations, comprising:
determining a type of undesired vibration compensation to apply to said audio signal;
retrieving an acoustic anti-transient-masking transform associated with said type of undesired vibration compensation; and
multiplying said audio signal by said acoustic anti-transient-masking transform to generate an output signal compensated for effects of said undesired vibrations.

19. The method as recited in claim 18 wherein said type of undesired vibration compensation is selected from the group consisting of:
a recording undesired vibration compensation,
a post-recorded undesired vibration compensation,
an audio component undesired vibration compensation, and
a sound amplification undesired vibration compensation.

20. The method as recited in claim 18 wherein said determining said type of undesired vibration compensation includes:
employing multiple acoustic anti-transient-masking transforms having various types of undesired vibration compensations;
obtaining a sample of said audio signal; and
determining which one of said multiple acoustic anti-transient-masking transforms produces the greatest dynamic range for said sample;

21. The method as recited in claim 18 wherein said multiplying said audio signal includes employing an amplitude offset, a phase offset, or a null spacing percentage to adjust said acoustic anti-transient-masking transform.

22. The method as recited in claim 18 wherein said method is performed in a batch mode.

23. The method as recited in claim 18 wherein said determining, said retrieving and said multiplying is performed for a plurality of undesired vibration compensations.
